(19) United States
(12) Patent Application Publication
(10) Pub. No.: US 2018/0358124 A1
(43) Pub. Date: Dec. 13, 2018

(54) Healthcare Reminder Label System With Visual Indicator

(71) Applicant: Precision Dynamics Corporation, Valencia, CA (US)

(72) Inventors: Adam Scheuer, Fox Point, WI (US); Heather Julian, Saugus, CA (US); Douglas Friedman, Simi Valley, CA (US); Paloma Velez, Valencia, CA (US)

(21) Appl. No.: 16/005,176

(22) Filed: Jun. 11, 2018

Related U.S. Application Data
(60) Provisional application No. 62/517,843, filed on Jun. 9, 2017.

(51) Int. Cl. G16H 40/60

(52) U.S. Cl. CPC G16H 40/60

(57) ABSTRACT

A reminder label system for attachment to a healthcare item and/or a patient. The reminder label system includes a label and a visual indicator. The label has pre-printed text corresponding to the healthcare item and/or the patient and further has a writeable surface to receive unique information about the healthcare item and/or the patient. The visual indicator is on the label and provides information relating to an event associated with the healthcare item and/or the patient. The visual indicator could provide any one of a number of different types of information including, but not limited to, time passage since activation of the indicator and exposure conditions of the indicator (e.g., temperature, radiation, sterilization, contamination, and so forth). Such a reminder label system can facilitate maintenance and patient care because it is detailed, secure, automatic, compliant, and workflow efficient.

### CROSS-REFERENCE TO RELATED APPLICATION

This application claims the benefit of U.S. Provisional Patent Application No. 62/517,843 filed on Jun. 9, 2017, the contents of which are incorporated by reference for all purposes as if set forth in their entirety herein.

### FIELD OF INVENTION

This disclosure relates to a reminder label system with a visual indicator in which the label system provides both preprinted or writeable information as well as the visual indicator.

### BACKGROUND

Maintenance is required of many items used in a healthcare setting. Currently, analog labels are applied to healthcare items and are used to help staff determine when maintenance is due, to track chain of contact in case of a health or security issue, and/or determine when and whether an item has been sterilized. These analog labels contain fields for receiving printed information such as time, date and registered nurse (RN) initials. These analog labels require that someone reach and read the label up close in order to gather any information from the label. This close inspection is inconvenient and costly because the practice of reaching and reading each individual label is a laborious, time intensive process.

### SUMMARY

To overcome the deficiencies with conventional analog labels for healthcare, one or more visual indicators are combined with labels having pre-printed text and fields for receiving patient-specific information or information relating to a particular item of medical equipment to which the label is attached. In many instances, the visual indicator may provide information relating to the passage of time since the indicator was activated. However, the indicator(s) are not so limited and could relate to time events and/or or other conditions such as, for example, temperature exposure, sterilization/contamination criterion, and radiation exposure. Such a reminder label system can facilitate maintenance and patient care because it can provide detailed and unique information, can be secure, can better ensure compliance, and can improve workflow efficiency.

According to one aspect, a reminder label system for attachment a healthcare item and/or a patient is provided. The reminder label system includes a label and a visual indicator. The label has pre-printed text corresponding to the healthcare item and/or the patient and further has a writeable surface to receive unique information about the healthcare item and/or the patient. The visual indicator is on the label and provides information relating to an event associated with the healthcare item and/or the patient.

In some forms, the visual indicator may indicate a passage of time since an activation of the visual indicator.

In some forms, the visual indicator may provide a continuous indication of the amount of time that has passed since activation and before a pre-established expiration time. In other forms, the indication may be “binary” in nature—for example, having one indication or appearance if a pre-established threshold time has not passed and a different indication or appearance if the pre-established threshold time has passed.

In some forms, the visual indicator may provide information relating to one or more of time passage, temperature exposure, radiation exposure, sterilization exposure, and contamination exposure. While this list of information provided by the indicator or indicators is exemplary, it is not to be considered limiting and other types of information might also be provided by the visual indicator or indicators.

In some forms, the label may support a plurality of visual indicators. Put differently, while the system is contemplated as being workable with just a single visual indicator on the label, it is contemplated that there could be more than one visual indicator each providing different kinds of information or even a single indicator that provides more than one type of information.

In some forms of the reminder label system, the reminder label system may include a set of visual indicators separately attachable to the label in which each of the set of visual indicators are configured to indicate the passage of time to a corresponding pre-determined expiration time. At least some of the set of visual indicators can have a different pre-determined expiration time than others to provide a set of pre-determined expiration times. By attaching a selected one of the set of visual indicators to the label, a visual indicator can be provided with a desired one of the set of pre-determined expiration times. In such a system, the set of visual indicators can include subsets of visual indicators having similar pre-determined expiration times in which each of the subsets have a unique coloration from the other subsets. In this way, an end user may quickly be able to identify both the expiration duration of the indicator (by seeing the coloration of the indicator itself) as well as whether such expiration duration has passed (by looking at a region of the indicator associated with expiration and/or time progression since activation).

In some forms, the visual indicator may be adhesively affixed to the label, for example, by an end user. However, it is contemplated that in other forms, the visual indicator may be wholly integrated into the label.

In some forms, the label itself may have adhesive on it for attachment of the label to the healthcare item and/or the patient. For example, it is contemplated that the label may be adapted for reception on an IV tube by having adhesive on one end thereof such that the end of the label having the adhesive thereon is wrappable around an IV tube to adhere the label to itself and secure the label to the IV tube. However, in other cases, the label might be attached to the healthcare item and/or patient by an adhesive on one side of the label or by other non-adhesive attachment structures. In still other forms, the label could have a head end receivable through a tail end to create an attachment loop.

It is contemplated that these attachment mechanisms—both of the label to the healthcare item and/or patient and, when applicable, of the visual indicator to the label—may incorporate security mechanisms to prevent tampering. For example, there could be a sacrificial layer or destructive layer that adheres to an adhesive such that, if the label or visual indicator is de-adhered or removed and replaced on another label, item and/or patient, such tampering will be visually evident.

In some forms, the pre-printed text on the label may include prompts for entry of a start date, end date, and registered nurse initials on the writeable surface. Likewise, in some forms, the pre-printed text may include at least in part a calendar text with months and dates which may be markable for selection. Such information can be helpful in documentation and compliance in the medical context.

In some forms, the label may be integrated into a medical bandage and a dressing.

In some forms, the label may be adapted for use in an application selected from a group of applications including attachment to an IV tube, attachment to a urinary catheter, attachment to equipment, attachment to a dressing, and attachment to a scope. This list is not all-inclusive, but is merely a representative list of some possible applications.

According to another aspect, a method of using a reminder label system of the type described above is contemplated. The method includes writing unique information on the writeable surface of the label about the healthcare item and/or the patient and attaching the label to the healthcare item and/or the patient. After attachment, information relating to an event from a visual indicator on the label is observed in which the event is associated with the healthcare item and/or the patient.

In some forms, the method may further include the step of activating the visual indicator. It is contemplated that, for certain types of indicators, the information relating to the event from the visual indicator may be time elapsed since the activation of the visual indicator and the activation step may initiate the timing event.

In some forms, the method may further include the step of attaching the visual indicator to the label. In some instances this may involve selecting the visual indicator from a set of visual indicators having subsets of visual indicators each having a pre-determined expiration time which is different than the other subsets. In this way, the visual indicator may be selected to have the desired time duration.

According to yet another aspect, a method of use of a label as described herein in a healthcare context is contemplated in which the visual indicator on the label communicates information in which the information relates to an event corresponding to the healthcare item and/or the patient.

While various structures and methods have been described separately and in combination in this disclosure, it will be appreciated that the disclosure is not limited to merely these specific descriptions. Any and all workable combinations and permutations of described features and steps are contemplated as being usable together to provide structures and methods incorporating different aspects of this disclosure.

These and still other advantages of the invention will be apparent from the detailed description and drawings. What follows is merely a description of some preferred embodiments of the present invention. To assess the full scope of the invention the claims should be looked to as these preferred embodiments are not intended to be the only embodiments within the scope of the claims.

### BRIEF DESCRIPTION OF THE DRAWINGS

FIG. 1 shows a reminder label system possessing pre-printed text and blank fields for receiving patient-specific information along with a generic visual indicator.

FIG. 2 shows a reminder label system similar to that of FIG. 1 having a color changing indicator (A) before activation and (B) after activation and beyond the expiration time.

FIG. 3 shows a reminder label system similar to FIG. 2, but in which the visual indicator is a progression indicator showing how the progression indicator appears (A) before activation, (B) 1 day after activation, (C) 2 days after activation, and (D) 3 days after activation and after expiration of the label.

FIG. 4 shows a reminder label system similar to that of FIG. 3 but which has been further adapted to have a wrap-around portion for attachment to IV tubing both (A) prior to attachment and (B) after attachment to the IV tubing.

FIG. 5 shows a reminder label system for equipment similar to that of the reminder label system of FIG. 3 except that, instead of the label including pre-printed text and blank fields for receiving patient-specific information, the pre-printed text and blank fields on the label of FIG. 5 are changed to relate to preventative maintenance information for an equipment maintenance reminder and to include longer indication intervals.

FIG. 6 shows a reminder label system configured for tracking time intervals (A) prior to activation and (B) 4 days after activation.

FIG. 7 shows a reminder label system that additionally provides marking areas for document compliance.

FIG. 8 shows a reminder label system in which the label and the indicators are shown as separate parts before assembly and in which the various indicators that may be placed on the label at the time of manufacture each are configured to track a different length of time (i.e., 24 hours, 48 hours, 72 hours, and 96 hours).

FIG. 9 shows the reminder label system of FIG. 8 in which four unique labels have each had a different time-length indicator attached thereto during manufacture in (A)-(D).

FIG. 10 shows a reminder label system with a generic indicator for a smart scope with a calendar feature and an alternate attachment structure for a looped attachment (A) before activation and (B) after activation;

FIG. 11 shows a reminder label system for a smart scope similar to FIG. 10 except that the generic indicator has been replaced with a specific time indicator having a 4-day expiration period shown (A) before activation and (B) after activation.

FIG. 12 shows a label system for an IV dressing tag in which the indicator is integrated onto a medical bandage/dressing.

FIG. 13 shows a label system where the indicator progresses vertically and is placed on the label at the time of activation.

### DETAILED DESCRIPTION

In the sections below, similar reference numerals will be used to describe features common among the various embodiments. For each different embodiment, a different 100's series will be used to denote the different embodiment (i.e., a first reminder label system 100 according to a first embodiment, a second reminder label system 200 according to a second embodiment, and so on) with common numbers being used to describe common features (i.e., feature 102 in the first embodiment will correspond to feature 202 in the second embodiment and so forth).

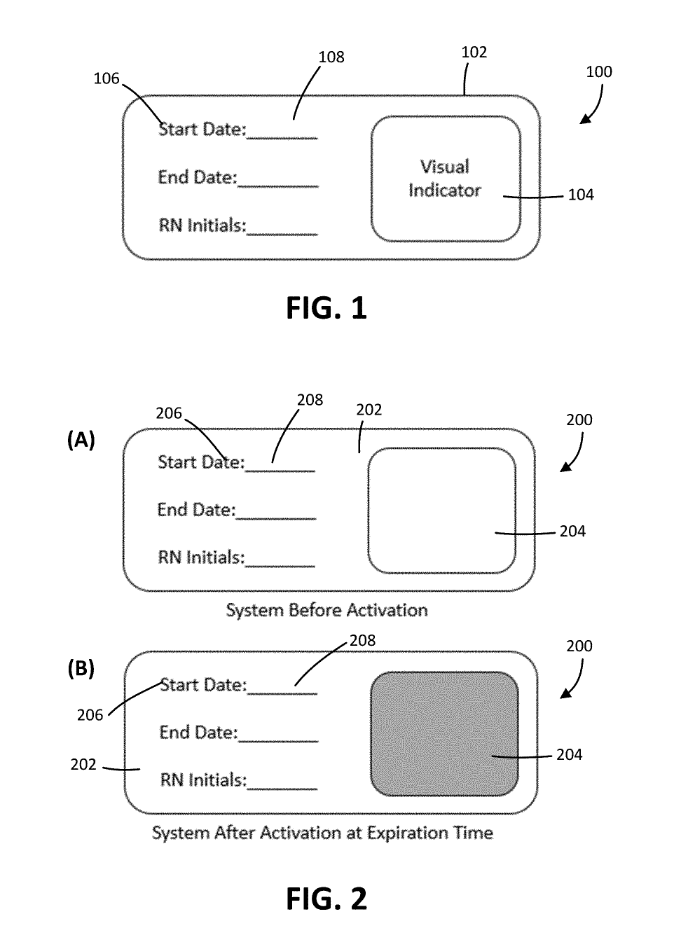

Referring first to FIG. 1, a reminder label system 100 is illustrated having a label 102 with a visual indicator 104 thereon. The reminder label system 100, which is initially presented in general and generic terms in this first schematic embodiment, will be generally described. Subsequent figures and embodiments will be used to express some exemplary variations and additional features that are contemplated as being employable in such reminder label systems.

Looking at the reminder label system 100, on one end thereof, the label 102 includes pre-printed text 106 thereon which corresponds to prompts for information related to an associated patient and/or healthcare item. Such information can be written by an end user or printed by a printing device on a writeable surface 108 of the label 102 in response to these prompts. In the label 102, the pre-printed text 106 specifically prompts for the entry of the start date, end date, and initials of a registered nurse. This information is associated with the usage of the reminder label system 100 and can be used to track when a particular associated event such as, for example, the passage of time has occurred to establish when a particular action (such as replacement of the healthcare item or an action to be performed on the patient) for the particular patient and/or healthcare item needs to be executed. However, other prompts associated with a particular healthcare use case might be used in place of the pre-printed text 106. As some non-limiting examples, the pre-printed portion of the label system may include a graphics such as calendar(s), check boxes, barcode(s), text, image(s), informational prompts, and so forth which may be in black and white and/or in color. The write-on portion or writeable surface 108 of the label system 100 may include adequate space for writing by the end user. In some forms, the write-on portion may also include a carbon copy means and/or may include an overlay shield to protect the writing from liquids and abrasion after the writing has occurred.

To provide a visual indication with respect to the state of an event of interest associated with the system 100, the visual indicator 104 is provided on the label 102. While the visual indicator 104 is generically illustrated in FIG. 1, the visual indicator 104 may take many physical forms, some of which are described in more detail herein. This visual indicator 104 may be separate from the label 102 and selectively placed on the label 102 (for example, by the application of the visual indicator 104 on the label 102 by the end user) or may be integrated with the label 102 such that the visual indicator 104 and the label 102 are initially constructed as a single and unitary structure before the system 100 is received by the end user.

The visual indicator 104 is generally capable of taking on two or more physical appearances—whether discrete or continuous in form—in order to indicate the state of an event associated with the visual indicator 104 and, by virtue of the visual indicator 104 being on the label 102, the state of the event as associated with the reminder label system 100. For example, the visual indicator 104 may be temperature-sensitive and may take on a first appearance (e.g., be white) when the visual indicator 104 has never exceeded a threshold temperature and then take on a different second appearance (e.g., be permanently red) after the threshold temperature has been exceeded. While temperature sensitivity is used as an example, visual indicators could be adapted to determine any number of events (for example, passage of time, temperature exposure, time-temperature exposure, radiation exposure, sterilization exposure, contaminate exposure, and so forth).

In some forms, the indicator technology may be a progression indicator and/or may be a chemical, electrochemical, or piezoelectric based process such as wicking, diffusion, etching, and so forth. The indicator may communicate an event by a color change, graphic, or other noticeable visual change. As noted above, the indicator may be a part of the label or a stand-alone item that is separately attached to create the label system.

Still yet, there may be activating actions associated with starting for the monitoring of the event by the visual indicator, as may be the case with monitoring of the passage of time. For example, the end user might apply pressure to a region of the visual indicator to start, for example, a chemical reaction or diffusive migration. As another example, if the user is assembling the visual indicator to the label, then it is contemplated that the action of assembly, attaching, or contacting the two may serve to activate the tracking or monitoring of the event by any one of a number of mechanisms (for example, chemical reaction or initiating migration or diffusion of a chemical species which may eventually react with another and so on). Indeed, this may occur via any assembly of layers. As another example, when an overlaminate or shield is used, that layer may be involved in activation when it contacts or is separated from another layer. Still yet, it is contemplated that in certain structures, the visual indicator might be initiated by an electric signal. Other forms of activation may be used in addition to those described above or the activation could be multi-factor in nature (e.g., requiring multiple actions to initiate activation).

In the case of time indicators, time-indicating labels, their structures, and methods of making them are widely understood by those having skill in the art and will not be described in great detail herein. For example, U.S. Pat. Nos. 4,212,153; 5,446,705; 5,633,835; 5,930,206; 5,974,003; 7,215,604; 8,999,262; and 9,110,446 each teach various time indicator labels and improvements thereto. Some labels use a visual time indicating technology that relies upon the diffusion of a dye through an opaque substrate to visually indicate the length of time since the activation of the particular label. Improvements on this dye diffusion technique to indicate time have been made, such as wicking of a colored fluid, acid etching of an opaque metal layer, fluid migration along a conduit, or electronic timer with display.

Such time-indicating labels can provide visual displays that can be instantly scanned from afar, allowing groups of tags to be observed from across a room in an instant. In the context of the healthcare environment, such long distance visibility provides great advantage because it may avoid the necessity of a registered nurse or caretaker reaching across the patient or needing to travel close to the label to view the written information on the label, as would have been traditionally necessary to establish the date and time information associated with such a label.

It is also worth noting that, conventionally, the visual indication on such time-indicating labels were the sole means of indicating the amount of time elapsed since activation of the time indicator. Put differently, there was not additional information, much less information specifically related to a healthcare item and/or to a patient. Accordingly, other visual indicators such as temperature, time-temperature, sterilization, radiation, freeze/thaw indicators typically only indicate a single piece of information relating to the state of the indicator, and often were not accurate or reliable enough to fully substitute for the documented information recorded on a preprinted/written label. Accordingly, while visual indicators in various forms have existed and are used in combination with the pre-printed text and the writeable surface herein to be functionally related and capable for use in a medical context, such labels historically had lacked the ability to receive and display written information in addition to providing the visual indicator. As healthcare protocol often necessitates written information on a reminder tag—because it must contain precise timing information, information on who provided maintenance last, or other such information—conventional labels and tags were not suitable for use because they failed to meet the necessary compliance and recordation standards.

Before proceeding to describe various additional specific embodiments with reference to FIGS. 2-13, some general usage and construction considerations will be discussed. Again, these variations are considered non-limiting and are provided to express some of the various structures that might be employed in the design of these reminder label systems.

With respect to the label 102, a label may typically be a paper/plastic film in the range of 0.5 mils-50 mils that is cut to a specific shape. As noted above, the label 102 may incorporate a protective film or overlaminate and, in some forms, the film or overlaminate may not be in place until the information has been written or printed on the writeable surface 108 of label 102. Further yet, it is contemplated that the protective film or overlaminate, when applied after the writing or printing is executed, may actually be used to activate the visual indicator 104 if the film or overlaminate contains an activating chemical, for example. Still yet, in some forms, it is contemplated that the temporary removal of a film or overlaminate could cause activation of the visual indicator 104 by exposing internal layers to the surrounding environment initiating an activating action.

The label system 100 is typically intended to be attached to a surface, an object, and/or a person. In some cases, the label system 100 may be applied directly to a person's skin or to an article associated with the person. The object may be a piece of medical equipment such as, for example, a medical scope, a pump, a tray, an IV bag, an IV line, a urinary catheter, a dressing/bandage such as a central line dressing, a surgical tool, a container, and so forth. Accordingly, the label 102 may include one or more modes of attachment that is integrated in the label 102. For example, with reference to FIG. 1, the reverse face of the label 102 from that illustrated can be adhesive coated and further have a removable release liner that covers the adhesive prior to use. In some forms, the label may have adhesive on at least a portion of the label for attachment to something such as an IV. It is contemplated that various modes of attachment could be used and the label may be at least partially attached via self-adhesion, a secondary adhesive, Velcro®, magnetism, clips, snaps, static cling, screws, nails, other fasteners, and any mode of attachment. It is contemplated that in some forms, the label may fold over around an article and adhere to itself, forming a tag or that it may be structured to form a loop without any kind of adhesive or binding contact.

It is contemplated that the label 102 may be antimicrobial and/or may be sterile.

The label 102 may also take on various form factors. For example, a label with adhesive could take the form of a bandage. The visual indicator 104 may be integrated into the bandage or attached to a bandage. An example of a bandage can be an IV dressing and, in many instances, IV dressing patches may have clear adhesive portions for attachment to the skin of a patient. The visual indicator 104 may be attached to other items that are associated with a patient. Still yet, the label 102 may be elongated in order to allow it to be used as a band.

In use, an end user writes or prints information upon the writeable surface 108, activating the visual indicator 104 (when the visual indicator 104 requires activation), and applies the label system 100 to the surface, object or person. However, the steps not necessarily occur in that order and, in fact can often occur in any order. For example, the label system 100 could be attached before writing occurs or activation occurs. Still yet, after attachment, the end user can periodically view or observe the reminder label system 100 to monitor for changes in appearance indicative of the event that the visual indicator 104 is adapted to indicate, track, and/or monitor.

Again, this reminder label system may be modular with the label portion and the indicator portion being provided separately and the end user may create the label system by joining the indicator to the label. As a non-limiting example, a nurse may want to add a secondary indicator to an existing label in use which may already have a first indicator. A nurse may need to track different events necessitating the application of subsequent indicators at different times. In another use scenario, a nurse may need to track several things simultaneously and apply various types of indicators at the same time. In yet another example, a nurse may need to re-start timing and apply a new time indicator over or next to the previous indicator.

We now turn to the remaining figures, FIGS. 2-13, which describe additional specific designs and use cases.

Looking at FIG. 2, a reminder label system 200 is illustrated which is nearly identical to the reminder label system 100 in FIG. 1, except that the visual indicator 104—which is generic in FIG. 1—is replaced with a specific color change indicator 204 in FIG. 2. As can be seen by comparing FIG. 2A (which shows the system 200 before activation) and FIG. 2B (which shows the system 200 after activation and expiration), the color change indicator 204 has shifted from a first color—white—in FIG. 2A to a different color—grey—in FIG. 2B. Obviously, the specific colors are for purposes of example only and these could be other colors. However, the example of FIG. 2 illustrates the general case of a binary-type visual indicator 204 in which the visual indicator 204 takes one of just two general appearances to show the status of an event. Although there are two general appearances, there still may be a gradual transition between the two appearances or states that occurs over a relatively brief period of time with respect to the event being tracked. This example does not get into the specific manner in which activation has occurred, although a specific form of activation is not necessary to understand the general principle of indication.

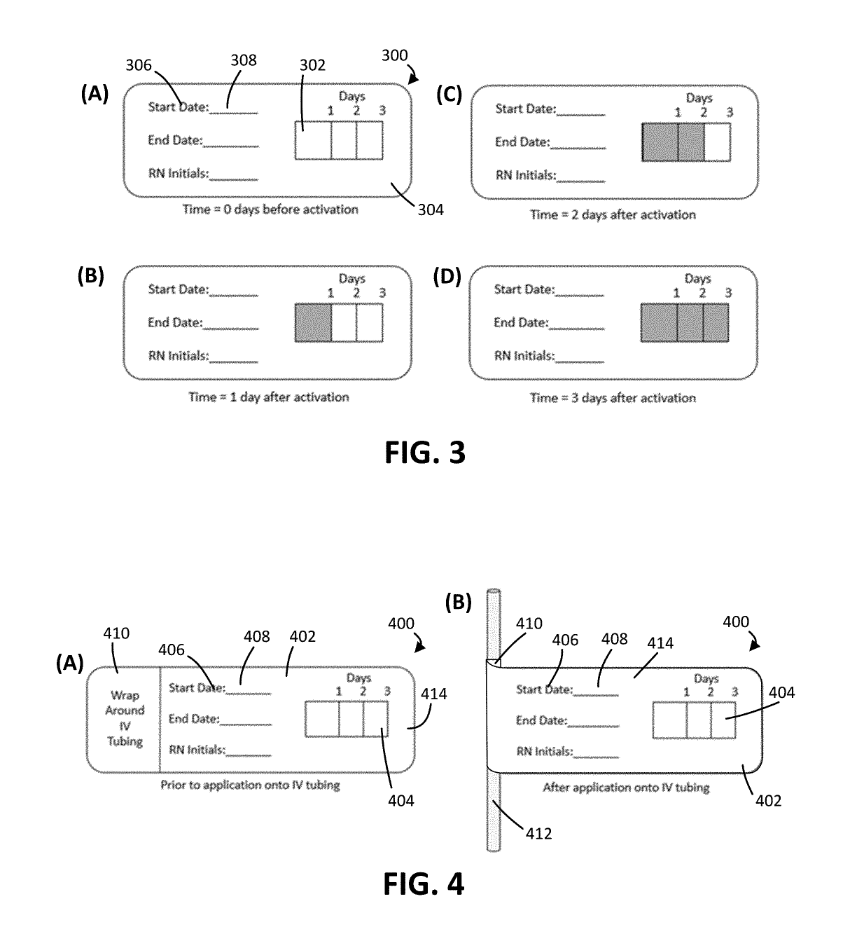

FIG. 3 illustrates a variation on the embodiment in which the reminder label system 300 has been designed to provide a visual indicator 304 that is a horizontally-aligned progression indicator with separate boxes indicating when zero, one, two, and three days have passed since activation (these are respectively indicated in FIGS. 3A, 3B, 3C, and 3D). In this way, the visual indicator 304 is not merely binary as in the example found in FIG. 2, but can provide a relative and progressive indication of the time that has passed. While it is noted that each of the times within FIG. 3 are discretely indicated as filling up to the line associated with the day count, it should be appreciated that where such a progression indicator is based on diffusion or migration, that the colorized portion of the bar can continuously advance from one side to the other (e.g., progress from the left side to the right side in a non-stepwise fashion). Still yet, in some forms, the day indications may be discrete or step-wise if, for example, each of the boxes are simultaneously activated and simply have different time delays before coloration changes occur based on a rate of reaction.

Turning to FIG. 4, a reminder label system 400 is illustrated which is identical to that of FIG. 3, except that the label 402 includes an attachment portion 410 which is specifically adapted to be received on an IV tube or other tubular object. In this structure, the attachment portion 410 can have an adhesive located only on the portion of the label 402 opposite the face of the attachment portion 410 illustrated in FIG. 4A. This adhesive may be covered by, for example, a removable release liner prior to application. However, other attachment modes could also be employed including those described earlier. To apply the reminder label system 400, the attachment portion 410 is wrapped around the IV tube 412 to secure the attachment portion 410 of the label 402 to the IV tube 412 such that a flag portion 414 remains positioned radially outward of the IV tube 412. The flag portion 414 includes the pre-printed text 406, any information received on the writeable surface 408 by the end user, and the visual indicator 404 (which, as illustrated is a progression indicator similar to that of FIG. 3) such that all of this pertinent information remains visible even after attachment.

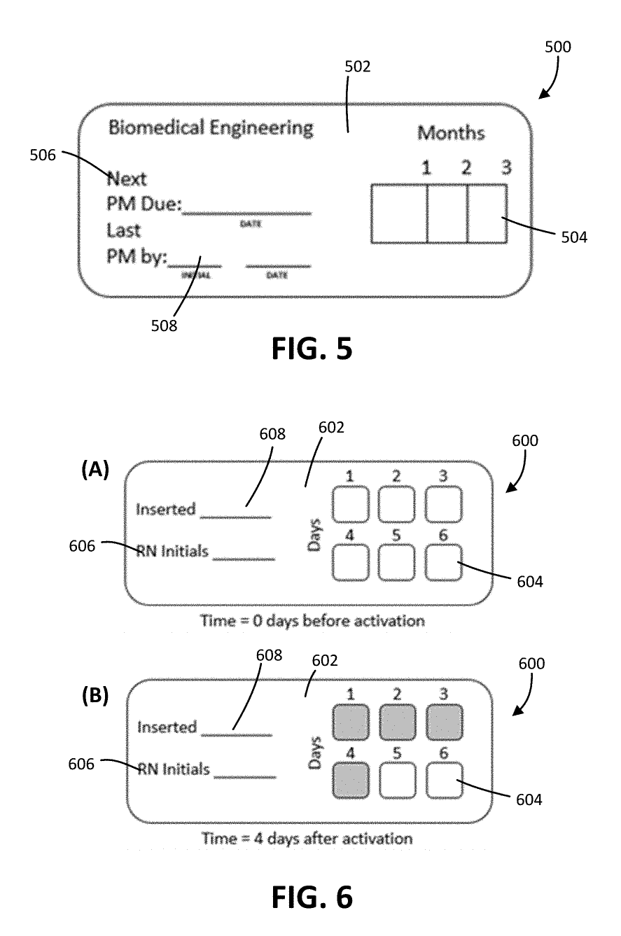

Turning now to FIG. 5, FIG. 5 shows a reminder label system 500 for an equipment maintenance reminder application. This system 500 has a similar appearance to the label system 300 from FIG. 3, but is adapted to the situation of equipment maintenance by (1) revising the pre-printed text 506 to correspond to preventative maintenance information (i.e., to include dates and by whom the last service was provided) and (2) by altering the visual indicator 504 to provide a visual indication after one, two, and three months—which in the case of the particular use case, preventative maintenance—is the appropriate time scale and is provided as a progression indicator. Of course, those periods of time are only by way of example and could be altered based on the time desired to be tracked based on the particular application case.

FIG. 6 shows a reminder label system 600 for tracking time intervals. However, in system 600, rather than having a progression bar as in the previous embodiments shown in FIGS. 3, 4, and 5, there are discrete and separate boxes corresponding to particular durations of time (specifically one, two, three, four, five, and six days). In this case, the likely construction of the system 600 involves designing the visual indicator 604 such that all of the plurality of boxes are simultaneously activated and the rate of reaction for color change in each box is engineered to be set to the corresponding time duration. Put differently, because this is a check box style configuration, each of the boxes will likely involve a separate time-controlled reaction to create a color change at their respective interval. By way of example, FIG. 6A illustrates the system 600 before activation, while FIG. 6B illustrates the system 600 four days after activation and two days before complete expiration of the system 600 (at which point, all boxes will have changed color).

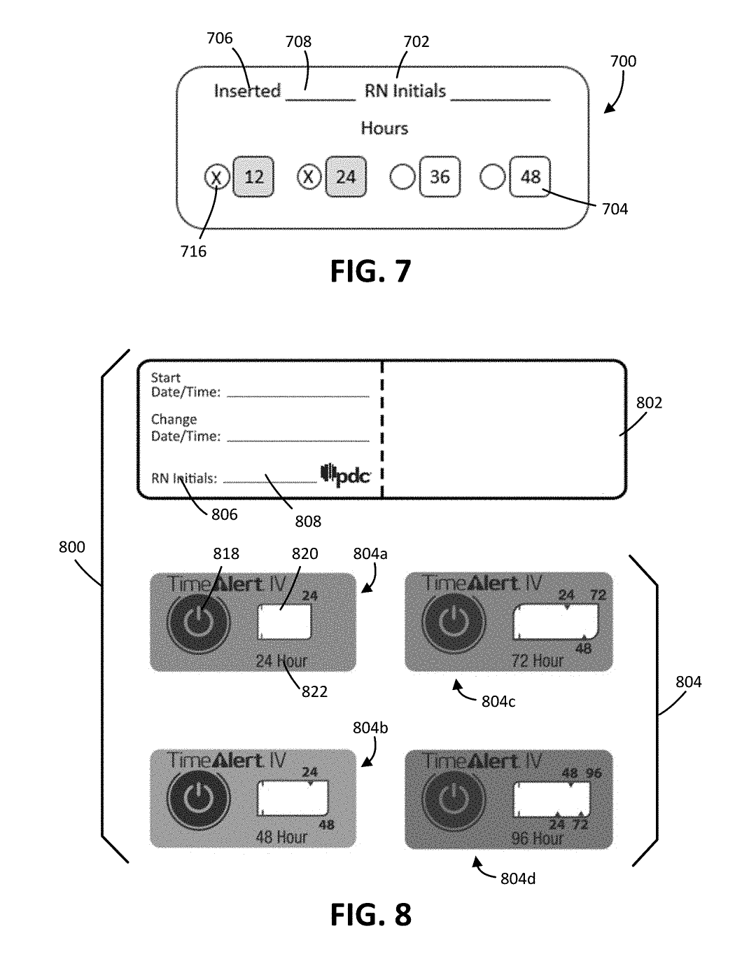

FIG. 7 shows a reminder label system 700 that additionally provides marking areas 716 for document compliance. In this reminder label system 700, the visual indicator 704 includes four discrete boxes each having a number of hours printed in them (12, 24, 36, and 48) to correspond with the number of hours that have elapsed since the activation of the system 700. Notably, just to the left of each of these boxes of the visual indicator 704 is a corresponding marking area 716 that is to be marked by the nurse or medical professional to ensure that the check has been performed.

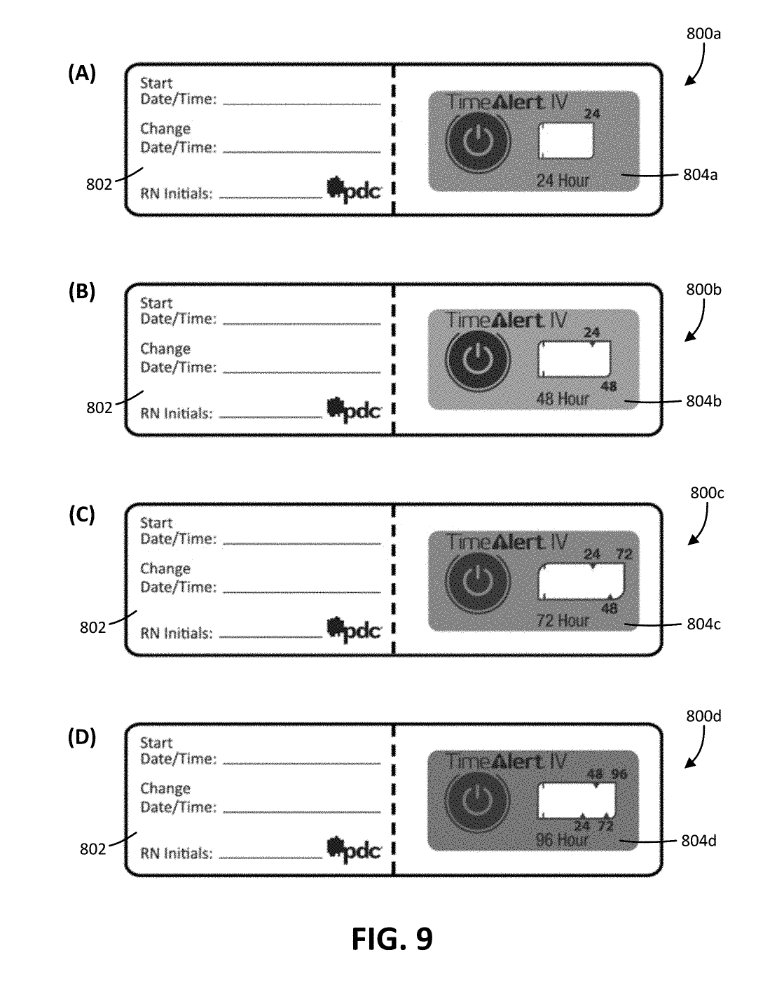

FIGS. 8 and 9 illustrate a reminder label system 800 in which one visual indicator is selected from a plurality of visual indicators 804 including different visual indicators 804a, 804b, 804c, and 804d and is then applied to the base label 802. The base label 802 includes the same pre-printed text 806 and writeable surface 808 in all instances on the left side of the label 802 and an open space on the right side of the label 802 for reception of any one of the visual indicators 804, while each of the visual indicators 804a, 804b, 804c, and 804d have a different expiration time associated with them (24 hours, 48 hours, 72 hours, and 96 hours respectively). Using visual indicator 804a as an example, each of the visual indicators 804 include an activation button 818 which may be a protruding pocket which upon depression initiates activation, a progression window 820 indicating the degree of expiration (which in the form illustrated, is a horizontal progression advancing from left to right which also has intermediate markings above and below to indicate the amount of intermediate time progression to expiration), and duration text 822 which indicates the expiration time of the respective visual indicator.

Each of the different visual indicators 804a, 804b, 804c, and 804d have a different color uniquely associated with the respective time duration so that the end user can, through repetitive use and association, know how long the indicator expiration duration is for the respective indicator by its color alone, even when the indicator is seen from a distance. Still further, the end user may also be able to see the progression window 820 from afar and, given both the unique color coding and the state of the progression bar 820, have a quick understanding of how long it has been since the particular visual indicator was activated.

Notably, in FIG. 8, the label 802 is shown apart from the visual indicators 804a, 804b, 804c, and 804d prior to any specific one of the visual indicators 804 being attached to the label 802. During manufacture, one of the various visual indicators 804 is selected and attached to the right side of the label 802 in the available space. This will effectively create one of the reminder label systems illustrated in FIG. 9. FIG. 9A illustrates a reminder label system 800a in which the visual indicator 804a has been attached to the label 802 to provide a 24-hour expiration period, FIG. 9B illustrates a reminder label system 800b in which the visual indicator 804b has been attached to the label 802 to provide a 48-hour expiration period, FIG. 9C illustrates a reminder label system 800c in which the visual indicator 804c has been attached to the label 802 to provide a 72-hour expiration period, and FIG. 9D illustrates a reminder label system 800d in which the visual indicator 804d has been attached to the label 802 to provide a 96-hour expiration period. In this way, great flexibility is provided for the construction and manufacture of a label system having the desired expiration time.

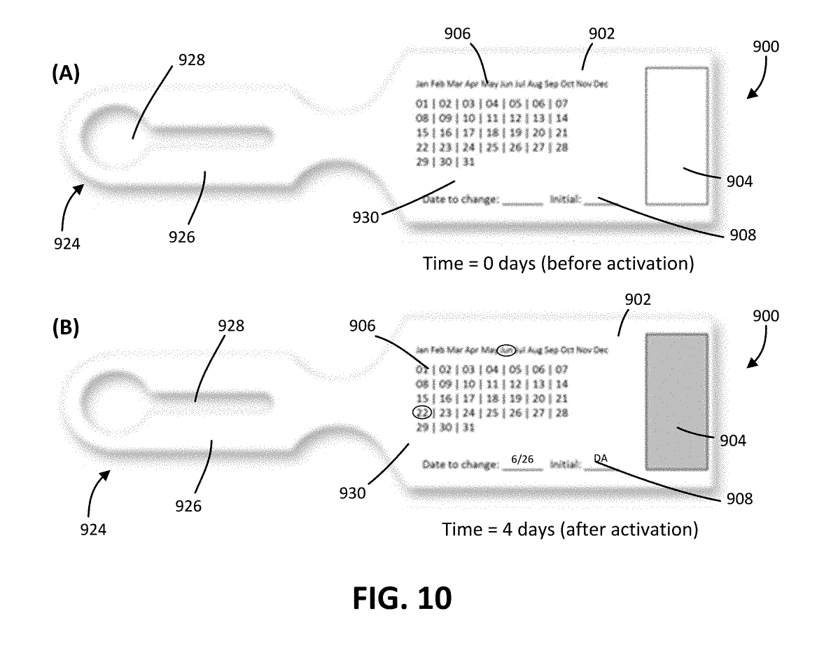

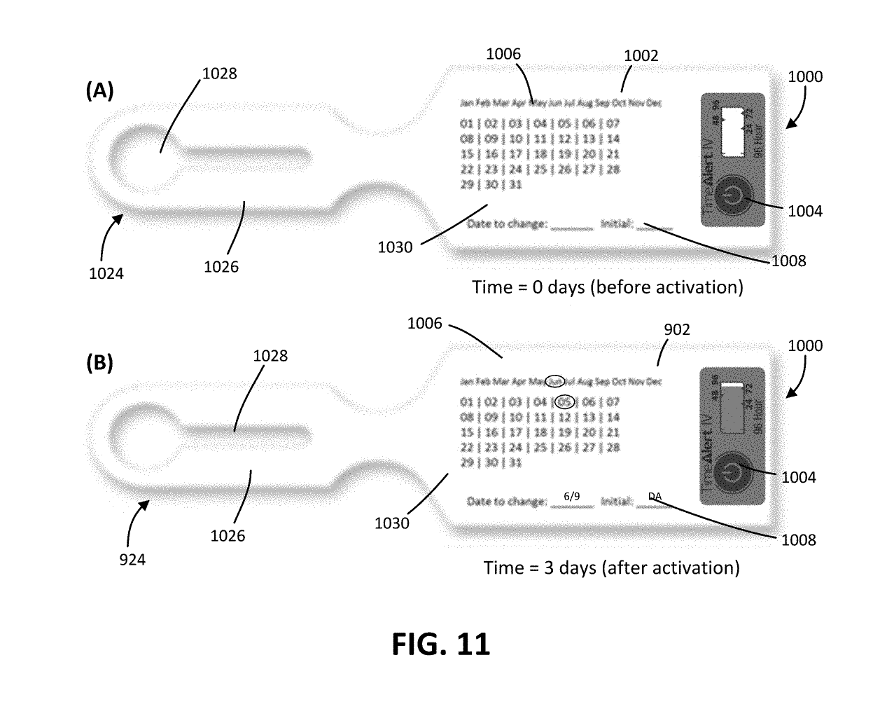

Referring now to FIGS. 10 and 11, two exemplary versions of a reminder label system 900 and 1000 are illustrated in which the systems 900 and 1000 incorporate a calendar feature as part of the pre-printed text 906 and 1006, respectively, and an alternate attachment structure 924 and 1024 for forming a looped attachment. The attachment structure 924 or 1024 includes a tail 926 and 1026 with an opening 928 and 1028 with a circle and a slot formed therein. These openings 928 or 1028 in the tail 926 and 1026 can accommodate passage of the head 930 or 1030 (i.e., the portion of the label 902 or 1002 including the text 906 and 1006 and visual indicator 904 and 1004) therethrough to form a loop which may be used to secure the label system 900 or 1000 around an object.

FIGS. 10A and 11A show the systems 900 and 1000 prior to activation of the respective visual indicator. FIGS. 10B and 11B show the same systems 900 and 1000 after the end user has marked the calendar feature to indicate a start date by circling the month and number date, filled in the expiratory date, and initialed and further illustrates the visual indicator 904 or 1004 after some amount of time from activation of the visual indicator 904 or 1004 has passed. In the case of FIG. 10B, the indicator 904 is a generic binary-type indicator that has passed its expiration time. In the case of FIG. 11B, the indicator 1004 has been attached as was the case in the system 800 shown in FIGS. 8 and 9, in which one of the indicators having the desired expiratory duration are attached to a common label.

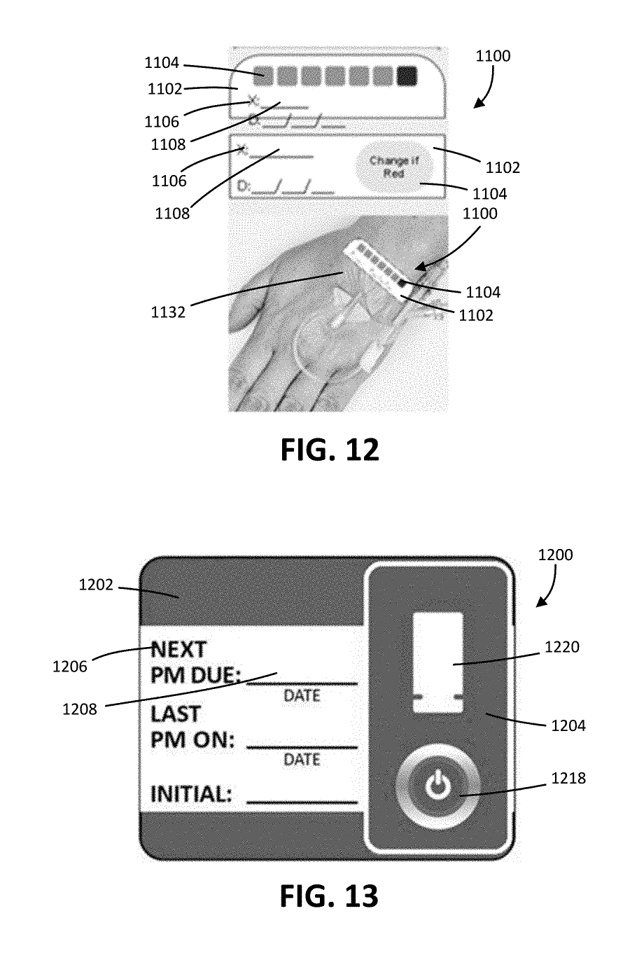

Referring now to FIG. 12, a reminder label system 1100 with two types of indicators 1104 (one having various separate boxes and one which is a single binary box) is illustrated in which the visual indicator 1104 is integrated into a medical bandage/dressing for retaining an IV in place. As can be seen, the label system 1100 most notably further includes an adhesive overlayer 1132 which extends beyond the edge of the opaque part of the label 1102 such that the label 1102 and system 1100 can be attached to the skin of a patient. The label 1102 can also be multi-part so that, for example, a part of the label is attached to the skin of the patient (as is shown in the bottom panel of FIG. 12) while the other part of the label may be attached elsewhere (such as, for example, to the IV fluid bag or other healthcare item).

Finally, with reference to FIG. 13, another reminder label system 1200 is illustrated in which the label 1202 itself can be of a desired color (along horizontal bands above and below the pre-printed text 1206 and the writeable surface 1208) along with the visual indicator 1204. The visual indicator 1204 also has a vertically-oriented progress window 1220 which shows progression after the activator button 1218 has been pressed. As with the color-coded system 800, the coloration on the reminder label system 1200 both on the label 1202 and the indicator 1204—when the color is consistently used and associated with an expiration time duration based on the color—can help provide the end user with a quick visual check on the length of the label and, by review of the progress window 1220, whether or not that expiration time has expired. It is contemplated that various color-coded labels may be used consistently in the same environment to make viewing the status of the label and knowing the expiration time easy.

To this end, with respect to coloration generally, color may be associated with a specific use and/or time limit in order to further simplify maintenance workflow. Colors may be green, purple, brown, and orange, for example. Specific uses for the indicator labels may be on IV tubing, urinary catheters, medical equipment, patient dressings, and scopes. For example, specific time limits may be relatively short for IV use cases (e.g., 24 hours, 48 hours, 72 hours, 96 hours, 7 days), relatively short for urinary catheters (e.g., 48 hours or 7 days), moderately long for smart dressings (7 days), or relatively long for smart equipment preventative maintenance labels (3 months, 6 months, 12 months). However, it will be appreciated that other use cases may have other lengths before expiration. As one example of color coding, a 24-hour IV tubing label can be green, while a 72-hour IV tubing label can be purple, and a 96-hour IV tubing label can be brown.

As noted above, it should be appreciated that various other modifications and variations to the preferred embodiments can be made within the spirit and scope of the invention. Therefore, the invention should not be limited to the described embodiments. To ascertain the full scope of the invention, the following claims should be referenced.

What is claimed is:

1. A reminder label system for attachment to at least one of a healthcare item and a patient, the reminder label system comprising:
a label having pre-printed text corresponding to at least one of the healthcare item and the patient and further having a writeable surface to receive unique information about at least one of the healthcare item and the patient; and
a visual indicator on the label in which the visual indicator provides information relating to an event associated with at least one of the healthcare item and the patient.

2. The reminder label system of claim 1, wherein the visual indicator indicates a passage of time since an activation of the visual indicator.

3. The reminder label system of claim 1, wherein the visual indicator provides a continuous indication of the amount of time that has passed before a pre-established expiration time.

4. The reminder label system of claim 1, wherein the visual indicator provides information relating to one or more of a group of information selected from time passage, temperature exposure, radiation exposure, sterilization exposure, and contamination exposure.

5. The reminder label system of claim 1, wherein the label supports a plurality of visual indicators.

6. The reminder label system of claim 1,
wherein the reminder label system comprises a set of visual indicators attachable to the label;
wherein each of the set of visual indicators are configured to indicate the passage of time to a corresponding pre-determined expiration time; and
wherein at least some of the set of visual indicators have a different pre-determined expiration time than others to provide a set of pre-determined expiration times;
whereby an attachment of a selected one of the set of visual indicators to the label provides the visual indicator with a desired one of the set of pre-determined expiration times.

7. The reminder label system of claim 6, wherein the set of visual indicators includes subsets of visual indicators having similar pre-determined expiration times in which each of the subsets have a unique coloration from the other subsets.

8. The reminder label system of claim 1, wherein the visual indicator is adhesively affixed to the label.

9. The reminder label system of claim 1, wherein the label has adhesive on it for attachment to at least one of the healthcare item and the patient.

10. The reminder label system of claim 1, wherein the label is adapted for reception on an IV tube by having adhesive on one end thereof such that the end of the label having the adhesive thereon is wrappable around an IV tube to adhere the label to itself and secure the label to the IV tube.

11. The reminder label system of claim 1, wherein the pre-printed text includes prompts for entry of a start date, end date, and registered nurse initials on the writeable surface.

12. The reminder label system of claim 1, wherein the pre-printed text includes a calendar text with months and dates.

13. The reminder label system of claim 1, wherein the label is integrated into at least one of a medical bandage and a dressing.

14. The reminder label system of claim 1, wherein the label is adapted for use in an application selected from a group of applications including attachment to an IV tube, attachment to a urinary catheter, attachment to equipment, attachment to a dressing, and attachment to a scope.

15. A method of using a reminder label system including a label having pre-printed text corresponding to at least one of a healthcare item and a patient further having a writeable surface, the method comprising:
writing unique information on the writeable surface of the label about at least one of the healthcare item and the patient;
attaching the label to at least one of the healthcare item and the patient; and
observing information relating to an event from a visual indicator on the label in which the event is associated with at least one of the healthcare item and the patient.

16. The method of claim 15, further comprising the step of activating the visual indicator.

17. The method of claim 16, wherein the information relating to the event from the visual indicator is time elapsed since activation of the visual indicator.

18. The method of claim 15, further comprising the step of attaching the visual indicator to the label in which the visual indicator is selected from a set of visual indicators having subsets of visual indicators each having a pre-determined expiration time which is different than the other subsets.

19. A method of using a reminder label system including a label having pre-printed text corresponding to at least one of a healthcare item and a patient and further having a writeable surface, the method comprising:
communicating information from a visual indicator on the label in which the information relates to an event corresponding to at least one of the healthcare item and the patient.
